(19) United States
(12) Patent Application Publication
(10) Pub. No.: US 2004/0029126 A1
(43) Pub. Date: Feb. 12, 2004

(54) Method For examining macromolecules

(76) Inventors: Helmut Bloecker, Braunschweig (DE); Gerhard Kauer, Braunschweig (DE)

Correspondence Address: Ronald R Santucci, Frommer Lawrence & Haug, 745 Fifth Avenue, New York, NY, 10151, US

(21) Appl. No.: 10/275,155

(22) PCT Filed: May 3, 2001
(86) PCT No.: PCT/EP01/05023

(30) Foreign Application Priority Data
May 5, 2000 (DE) 100-21-689.7

(51) Int. Cl. C12Q 1/68; G06F 19/00; G01N 33/48; G01N 33/50

(52) U.S. Cl. 435/6; 702/19; 702/20

(57) ABSTRACT

The invention relates to a method for examining macromolecules which can be stored in frequency-based data patterns. The invention also relates to a device for carrying out said method and to different applications of both the method and device. The method itself is based on: the compilation of sequence data of molecular sequences of macromolecules; the conversion of the sequence data into frequency-modulated frequency data; the transformation of the frequency data into a Fourier space; the use of Fourier analyses for comparing, weighting, cataloging and/or typifying the frequency data and, finally, for re-transforming the weighted, cataloged and/or typified frequency data into sequence data provided in a weighted, cataloged and/or typified form.

The invention relates to a method of investigating macromolecules and to an apparatus for carrying out the method in model manner and to uses of the method and/or of the apparatus according to the independent claims.

Within databases there have accumulated vast datasets in the form of sequence-based data samples for a very great variety of macromolecules. Such datasets are used for dealing with biological problems arising out of information within macromolecular sequence data. It is currently possible to deal with such problems only by using computer-assisted methods, the vast datasets requiring considerable computer power, especially as the ever increasing worldwide sequencing output from current and planned genome projects is experiencing an unexpectedly high degree of growth. As a result, the problem arises as to how to apply the available algorithms to the problems in question efficiently, without coming up against the limits of computing power.

The problem is solved by the subject-matter of the independent claims. Advantageous developments of the invention are described in the subordinate claims.

The method according to the invention for solving the above problem in investigating macromolecules accordingly comprises the following method steps:

a) establishment of sequence data of molecular sequences of macromolecules;

b) conversion of the sequence data into frequency-modulated frequency data;

c) transformation of the frequency data into a Fourier space;

d) use of Fourier analyses for comparison, weighting, cataloguing and/or typing of the frequency data;

e) back-transformation of the compared, weighted, catalogued and/or typed frequency data to form sequence data in weighted, catalogued and typed form.

The method makes possible an entirely new technology for the efficient analysis of vast sequence-based macromolecule datasets. The potential of this technology lies, firstly, in considerably increasing the speed of the macromolecule analyses in question and also in the possibility that entirely new information-gathering problems will be identified.

In a preferred embodiment of the method, a method of information filtering from digital image analysis is used for comparison, weighting, cataloguing and/or typing. This embodiment has the advantage that it is possible both to measure the similarity of two one-dimensional samples where there is respective positional displacement by i data points and also to search a signal with a specified signal trace, a measure of similarities being produced as a result of image analysis and it being possible, as a result, to draw conclusions with respect to similarities between the macromolecules. That similarity becomes maximal when the displacement produces maximal concordance between the sequence of frequency data and the sample. By means of that displacement, the unambiguous position of the one-dimensional sample in the frequency data sequence is also unambiguously given, by means of back-transformation and demodulation, by the position of the sample in a sequence.

The use of the Fourier transform simplifies detection filtering by means of the folding and, as a result, speeds up the investigation to a considerable degree.

In a further embodiment of the method, a method of frequency analysis is used for comparison, weighting, cataloguing and/or typing. In this embodiment, the sequence data, having first been converted into frequency-modulated data, are so processed that an unambiguous frequency datum is assigned to each element of a sequence in correlation to its neighbour. Although the actual sequence recedes into the background as a result and is, in the simplest case, transformed into a one-dimensional frequency-modulated wave, the sequence information is unaffected by that transformation and is merely converted into a complex frequency datum having the same information content. The advantage of this embodiment is that any mathematical method of frequency analysis can be applied to the frequency-modulated wave. In particular, spectral information analysis is of greatest benefit in this context.

In a further embodiment of the method, stochastic information filtering in the Fourier space is used for comparison, weighting, cataloguing and/or typing. In this embodiment, it is advantageously possible to estimate deviations from the ideal signal stochastically, as a result of which the expectation horizon can be formulated in dependence upon the biological problem.

In a further preferred embodiment of the method, the information units and/or structural information of multi-dimensional protein and/or DNA databases are encoded into corresponding sequence codes for establishing sequence data. It is advantageous therein that, when investigating macromolecules and biological problems relating to macro-molecules, it is possible to have recourse to multi-dimensional protein and/or DNA databases, which can then be appropriately evaluated and analysed using the method according to the invention without the limits of efficiency of the methods used and the considerable computing powers being surpassed.

The method according to the invention can be carried out preferably using an apparatus that comprises a large number of electronic modules for modelling frequency data that simulate molecular sequences and a large number of frequency filters for weighting, cataloguing and/or typing the frequency data modelled by the large number of electronic modules. A significant advantage of the method according to the invention is that it is readily possible, on the one hand, to develop the necessary algorithms and filter systems on a computer but thereafter to convert the methods found into electronic circuits and then to carry out the algorithms, no longer with computer assistance but rather in a high-frequency circuit. It is accordingly possible, using such an apparatus, to investigate interactively very large sequence-based datasets, for example entire genomes, quickly and virtually free of delay.

In a preferred embodiment of the apparatus, the large number of electronic modules and the large number of frequency filters are determined by means of computer-assisted frequency analyses and they are coupled up to one another to form a hardware network which simulates the sequence of information units of macromolecules. In this context, the information units are bases of nucleic acids, amino acid residues of proteins and/or DNA, the sequence of which in a macromolecule are simulated by the hardware network. This embodiment of the invention makes it possible not only to make a rapid comparison of large sequence-based data samples but also, in addition, by means of the macromolecule-modelling hardware network, to deal with biological problems directly at the speed of light and to answer them at correspondingly high speed.

The method and apparatus of the invention are preferably used for the analysis of protein sequences. Advantageously, uses in the context of the analysis of DNA sequences are likewise possible. For that purpose, investigations and samplings of multi-dimensional protein databases may also be used. For that purpose, the information units of the databases need to be provided in corresponding sequence codes, which may also be multi-dimensional. Consequently, it is not restrictively necessary to limit spectral analyses merely to one, two or three dimensions, especially as in the preferred uses the invention can be used for a large number of information fragments.

In a preferred use of the invention, multi-dimensional DNA structural information is investigated for repeating patterns. In particular, it is possible, using the invention, to investigate biological problems, interactively and free of delay, for sequence-based datasets.

The invention will be described in greater detail below with reference to exemplary embodiments.

In a first exemplary embodiment, the sequence data are first converted into frequency-modulated data. Each element of the sequence in correlation to its neighbour accordingly receives a unitary frequency datum. The actual sequence recedes into the background as a result and is, in the simplest case, transformed into a one-dimensional frequency-modulated wave. The sequence information is unaffected by that transformation and is merely converted into a complex frequency datum having the same information content.

The advantage of this method is that any mathematical method for signal processing can then be applied to the frequency-modulated wave. In particular, spectral information analysis provides the greatest benefit in this context.

A Fast Fourier Transform (FFT) is then applied to the frequency-modulated wave. Appropriate filters are then applied to the transformed data. After back-transformation, the so-called Inverse Fourier Transform (IFFT) and demodulation of the frequency data back into the sequence data, the appropriately filtered information is obtained.

Consequently, sequence samples can be searched very efficiently in the output spectrum, for example large portions of genome or entire genomes are compared with one another and filtered out. Deviations from the ideal signal can be estimated stochastically, it being possible for the expectation horizon to be formulated as desired in dependence upon the biological problem. That results in the significant advantage of the method according to the invention, namely that it is readily possible first to develop the necessary algorithms and filter systems on a computer and thereafter to convert the methods found into electronic circuits. The algorithms in question then no longer need to be processed in a computer but can be processed in a high-frequency circuit. Using this embodiment of the invention it is consequently possible to investigate interactively a very large sequence-based dataset, for example entire genomes, quickly and free of delay.

The method according to the invention is, however, not limited to the simplest case of a one-dimensional frequency-modulated wave. Rather, in a second example of an embodiment of the invention, it is also possible for three-dimensional or multi-dimensional protein databases or multi-dimensional DNA structural information to be investigated for corresponding patterns in entirely similar manner. For that purpose, databases will convert their information units into corresponding sequence codes. The method according to the invention can also be used for an assembly of a large number of n information fragments, as are present, for example, in “shotgun”-organised databases. The sum of those n information fragments constitutes the total information of a logic unit N, it being possible for the sum of all partial elements of the fragments to be substantially larger than the sum of partial elements of the total information N:

n>>N;∀{nεN}

Once the sequence information is available in frequency-modulated form, it is transformed, in accordance with the present invention, by means of a Fast Fourier Transform, wherein, in the simplest case, the correlation function φ_{fg} of two one-dimensional signals, namely f(m) and g(m), is to be interpreted as a folding of the signal f(m) with the signal (g-m).

$${{ϕ}_{fg} ( i )}_{n} = \sum f ( m ) g ( m - i )$$

Using this mode of operation, it is possible both to measure the similarity of two one-dimensional samples where there is respective positional displacement by i image points and also to search within a signal f(m) for a signal trace specified by g(m), φ_{fg} being the measure of the similarity. That measure becomes maximal when the displacement i produces maximal concordance between the wave f(m) and the sample g(m). By means of that displacement the unambiguous position of the one-dimensional “sample” in the wave is then given. By means of back-transformation and demodulation, the position of the sample in the sequence can be unambiguously determined. The FFT advantageously simplifies this detection filtering by means of the folding. The Fourier transformands Φ_{fg} and F are calculated from φ_{fg} and f and exhibit the following relation:

Φ_{fg} (k)=F(k)G*(k)

wherein G*(k) is the conjugated complex Fourier transformand of g(m). In the case of the present vast datasets of sequence-based data samples of macromolecules, the operation is in this instance advantageous in Fourier space, because extensive sample functions are already available for the problem being addressed. Exact concordance of f(m) and g(m) supplies for φ_{fg} the signal energy of f(m) and g(m).

As a third example, the following two-dimensional relations shall now be mentioned:

φ_{fg} (i,j)=ΣₘΣₙf(m,n)g(m−i,n−j), and Φ_{fg}(k,l)=F(k,l)G*(k,l)

In this context, detailed analyses of information-bearing biological macromolecules show that there is superimposed on the pure sequence information a considerable information content resulting from chemically related patterns of neighbouring modules or, for example, multi-dimensional location signals.

The methods described above by way of example for one-dimensional and two-dimensional relations can rapidly determine such additional information content by means of suitable stochastically acting filters in the frequency space.

As a result of suitable mapping of the relevant “similarity function” of involved modules or module groups into the frequency space, there are automatically produced structures which can be determined by proven filters. For example, analyses with local output spectra can be used which deal with the spectral energies of the portions to be investigated.

The output spectrum |F(k)|² is the Fourier transformand of the autocorrelation function of the signal f(m) and can therefore be used for measuring the statistical bonds between the values of neighbouring data of f(m). When the output spectra are calculated within local windows, it is also possible for samples that do not have a stationary location to be described as a result. A suitable weighting of the original function can be used in order to reduce disruptive components in the output spectrum. In digital image analysis, for original text detection before the Fourier transform, for example, a Hemming function of the following kind is used

$$h ( m , n ) = {\prod }_{i = m , n} ( 0.54 - 0.46 cos ( \frac{2 \prod i}{15} ) )$$

1. Method of investigating macromolecules, having the following method steps:
a) establishment of sequence data of molecular sequences of macromolecules;
b) conversion of the sequence data into frequency-modulated frequency data;
c) transformation of the frequency data into a Fourier space;
d) use of Fourier analyses for comparison, weighting, cataloguing and/or typing of the frequency data;
e) back-transformation of the compared, weighted, catalogued and/or typed frequency data to form sequence data in weighted, catalogued and/or typed form.

2. Method according to claim 1, characterised in that methods of information filtering from digital image analysis are used for the comparison, weighting, cataloguing and/or typing.

3. Method according to claim 1 or claim 2, characterised in that methods of frequency analysis are used for the comparison, weighting, cataloguing and/or typing.

4. Method according to one of the preceding claims, characterised in that stochastic information filtering in the Fourier space is used for the comparison, weighting, cataloguing and/or typing.

5. Method according to one of the preceding claims, characterised in that information units and structural information of multi-dimensional protein and/or DNA databases are encoded into corresponding sequence codes for establishing sequence data.

6. Apparatus for investigating macromolecules, having a large number of electronic modules for modelling frequency data which simulate molecular sequences, and having a large number of frequency filters for weighting, cataloguing and/or typing the frequency data modelled by the large number of electronic modules.

7. Apparatus according to claim 6, characterised in that the large number of electronic modules and the large number of frequency filters are determined by means of computer-assisted frequency analyses and are coupled up to one another, with computer assistance, to form a hardware network which simulates the sequence of information units of macromolecules.

8. Apparatus according to claim 7, characterised in that the information units are bases of nucleic acids, amino acid residues of proteins and/or three-dimensional structural units of proteins and/or DNA.

9. Use of the method according to one of claims 1 to 5 or of the apparatus according to claim 6, 7 or 8 for analysis of protein sequences.

10. Use of the method according to one of claims 1 to 5 or of the apparatus according to claim 6, 7 or claim 8 for analysis of DNA sequences.

11. Use of the method according to one of claims 1 to 5 or of the apparatus according to claim 6, 7 or 8 for investigating and sampling three-dimensional protein databases.

12. Use of the method according to one of claims 1 to 5 or of the apparatus according to claim 5, 6 or 8 for investigating three-dimensional DNA structural units for repeating patterns.

13. Use of the method according to one of claims 1 to 5 or of the apparatus according to claim 5, 6 or 8 for interactive investigation, free of delay, of sequence-based datasets of differently structured macromolecules.
